(19) United States
(12) Patent Application Publication
(10) Pub. No.: US 2026/0134972 A1
(43) Pub. Date: May 14, 2026

(54) Nutrition Evaluation and Recommendation Supported by Mitochondrial Enzyme Testing

(71) Applicants: Christopher Masterjohn, Miami, FL (US); Hannah Saxton, Miami, FL (US)

(72) Inventors: Christopher Masterjohn, Miami, FL (US); Hannah Saxton, Miami, FL (US)

(21) Appl. No.: 19/390,001

(22) Filed: Nov. 14, 2025

Related U.S. Application Data
(60) Provisional application No. 63/720,586, filed on Nov. 14, 2024.

(51) Int. Cl. G16H 20/60; G16H 10/40

(52) U.S. Cl. CPC G16H 20/60; G16H 10/40

(57) ABSTRACT

A nutrition recommendation system incorporating results from mitochondrial enzyme testing includes a data aggregation module arranged to receive mitochondrial enzyme testing test data from at least one of citrate synthase, Complex I, Complex II, Complex II plus Complex III, and Complex IV testing. A data processing and recommendation module receives and processes that test data, recommending at least one protocol comprising nutrient suggestions for mitochondrial health.

### RELATED APPLICATION

This application claims the priority benefit of U.S. Patent Application No. 63/720,586, filed on Nov. 14, 2024, which is hereby incorporated by reference in its entirety.

### TECHNICAL FIELD

The present disclosure relates generally to a recommendation system based on tested mitochondrial enzyme function. In certain embodiments, data derived from commercially available mitochondrial enzyme testing can be used to recommend various nutrients, adjustment of lifestyle factors, minimization of environmental toxicities, or medications.

### BACKGROUND

Mitochondria are cellular organelles that are present in most kinds of human cells and have a primary function of generating energy necessary to power cells. In mitochondria, the metabolism of sugars is completed, with the glucose sugar breakdown product pyruvate being imported into mitochondria and oxidized to carbon dioxide and water. As compared to anaerobic glycolysis, this type of respiration allows as much as fifteen times more adenosine triphosphate (ATP) to be made than that produced by glycolysis alone.

Mitochondrial function relies on enzymes and coenzymes that include those that metabolize pyruvate and fatty acids to produce acetyl CoA and those that oxidize acetyl CoA in the citric acid cycle. In mitochondria, enzymes are a part of a process that ensures electrons are transported along a respiratory electron-transport chain as a part of the oxidative phosphorylation process, generating most of the cell's ATP. Atypical mitochondrial enzyme activity can indicate nutritional deficiencies, toxin exposures, or genetic disorders. Systems and methods for identifying and recommending nutrients or other factors for improving mitochondrial health are needed.

### SUMMARY

A nutrition recommendation system incorporating results from mitochondrial enzyme testing includes a data aggregation module arranged to receive mitochondrial enzyme testing test data from at least one of citrate synthase, Complex I, Complex II, Complex II plus Complex III, and Complex IV testing. A data processing and recommendation module receives and processes that test data, recommending at least one protocol comprising nutrient suggestions for mitochondrial health.

In some embodiments, each of measurements Complex I, Complex II, Complex II plus Complex III, and Complex IV can be normalized to citrate synthase.

In some embodiments, each of measurements of citrate synthase, Complex I, Complex II, Complex II plus Complex III, and Complex IV can be expressed as percent of a mean of a control dataset.

In some embodiments, a Fix Complex I protocol is recommended by the data processing and recommendation module.

In some embodiments, a Fix Complex II protocol is recommended by the data processing and recommendation module.

In some embodiments, a Fix Complex II plus Complex III protocol is recommended by the data processing and recommendation module.

In some embodiments, a Fix Complex IV protocol is recommended by the data processing and recommendation module.

In some embodiments, a Feed Complex II protocol is recommended by the data processing and recommendation module.

In some embodiments, a Feed Complex I protocol is recommended by the data processing and recommendation module.

In some embodiments, a Peroxisome protocol is recommended by the data processing and recommendation module.

In some embodiments, a Methylation protocol is recommended by the data processing and recommendation module.

In some embodiments, an Iron-Sulfur protocol is recommended by the data processing and recommendation module.

In some embodiments, a Heme protocol is recommended by the data processing and recommendation module.

In some embodiments, a Cytoxan protocol is recommended by the data processing and recommendation module.

In some embodiments, a Feed Complex IV protocol is recommended by the data processing and recommendation module.

In some embodiments, a Mitochondrial Biogenesis protocol is recommended by the data processing and recommendation module.

In some embodiments, a Sulfur Metabolism Optimization protocol is recommended by the data processing and recommendation module.

In some embodiments, an Excessive Hypoxia protocol is recommended by the data processing and recommendation module.

In another embodiment, a nutrition recommendation method incorporating results from mitochondrial enzyme testing can include the steps of arranging a data aggregation module to receive mitochondrial enzyme testing test data from at least one of citrate synthase, Complex I, Complex II, Complex II plus Complex III, and Complex IV testing and using a data processing and recommendation module to recommend at least one protocol comprising nutrient suggestions for mitochondrial health.

### BRIEF DESCRIPTION OF THE DRAWINGS

FIG. 1 is a simplified illustration of selected enzyme complexes and components in a mitochondrial respiratory chain that results in usable energy for a cell.

FIG. 2 is an illustration of one embodiment of a recommendation system based on tested mitochondrial enzyme function.

FIG. 3 is a flow chart illustrating selected steps in a recommendation method.

### DETAILED DESCRIPTION

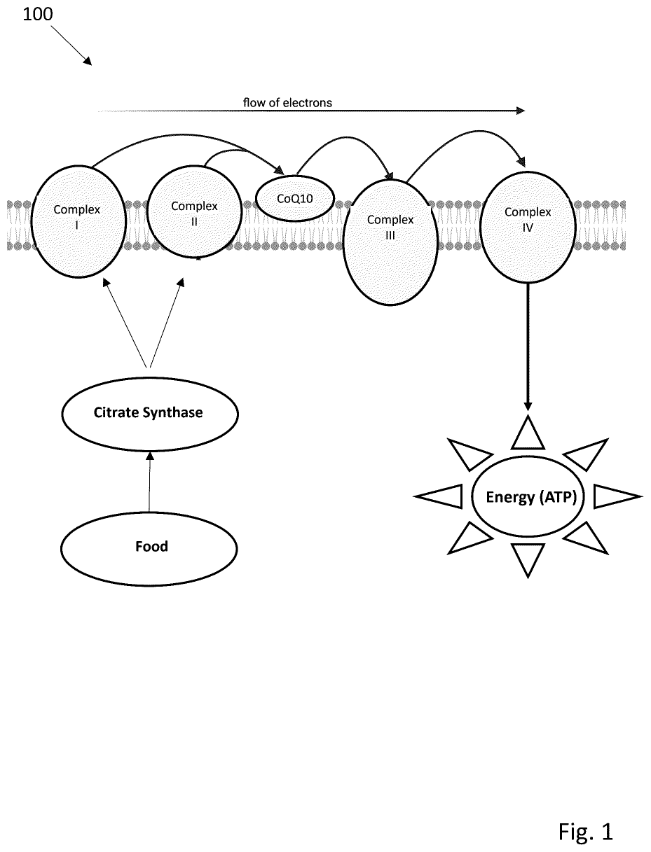

FIG. 1 is a simplified illustration of selected complexes and components in a mitochondrial respiratory chain that results in usable energy for a cell. As illustrated, Complex I, Complex II, Complex III, Complex IV, and CoQ10 are shown embedded in or attached to a mitochondrial lipid bilayer. Additionally, citrate synthase is associated with the mitochondrial matrix and acts on nutrients and food provided to the respiratory chain. Arrows indicate flow of electrons that ultimately result in cellular energy production through biochemical generation of ATP (oxidative phosphorylation).

Respiratory complex I (also known as NADH:ubiquinone oxidoreductase, Type I NADH dehydrogenase and mitochondrial complex I) is the first large protein complex of the respiratory chain. Complex I catalyze transfer of electrons from NADH to coenzyme Q10 (CoQ10) and translocate protons across the inner mitochondrial membrane. This enzyme is essential for the normal functioning of the respiratory chain in cells, and mutations or deficiencies in its operation can result in a range of neuromuscular or metabolic disorders.

Respiratory complex II (also known as succinate dehydrogenase (SDH) or succinate-coenzyme Q reductase (SQR)) is the second large protein complex of the respiratory chain. In the citric acid cycle, Respiratory complex II catalyzes the oxidation of succinate to fumarate with the reduction of ubiquinone to ubiquinol. This occurs in the inner mitochondrial membrane by coupling the two reactions together. This enzyme is essential for the normal functioning of the respiratory chain in cells, and mutations or deficiencies in operation can result metabolic control or inflammation issues.

Respiratory complex III (also known as coenzyme Q:cytochrome c-oxidoreductase, and sometimes called the cytochrome bc1 complex) is the third large protein complex in the electron transport chain. Complex III enzyme is essential for the normal functioning of the respiratory chain in cells, and mutations or deficiencies in operation can result in exercise intolerance or multisystem disorders.

Respiratory complex IV (also known as cytochrome c oxidase) is the fourth larges protein complex in the electron transport chain. It can receive electrons from respiratory Complex III via the mobile electron carrier cytochrome C and transfer them to an oxygen molecule with protons, producing water. In addition to binding protons from an inner aqueous phase, it transports another four protons across the mitochondrial membrane, increasing the transmembrane difference of proton electrochemical potential, which the ATP synthase then uses to synthesize ATP. This enzyme is essential for the normal functioning of the respiratory chain in cells, and mutations or deficiencies in its operation can result in a range of metabolic disorders, particularly in tissues and organs with high energy demands such as brain, heart, or muscle.

Coenzyme Q10 (also known as Coenzyme Q, CoQ, CoQ10, Ubiquinone, Ubiquinone-Q10, Ubidecarenone, or Vitamin Q10) is a cofactor in the electron-transport chain, in the series of redox reactions that are involved in the synthesis of adenosine triphosphate. This coenzyme is essential for the normal functioning of the respiratory chain in cells. Deficiency of coenzyme Q10 leads to the dysfunction of the respiratory chain, which leads to insufficient production of highly energetic compounds such as ATP, and results in decreased cell health.

Citrate synthase is an enzyme that acts on oxaloacetate and acetyl CoA to produce citrate, the first step of the citric acid cycle, which allows breakdown products of fatty acids, carbohydrates, and protein to be further broken down into carbon dioxide and high-energy electrons that can be delivered to the respiratory chain. Citrate synthase is located in the mitochondrial matrix and is commonly used as a quantitative enzyme marker for the presence of intact mitochondria. For example, activity of citrate synthase can act as a proxy for mitochondrial content of muscle.

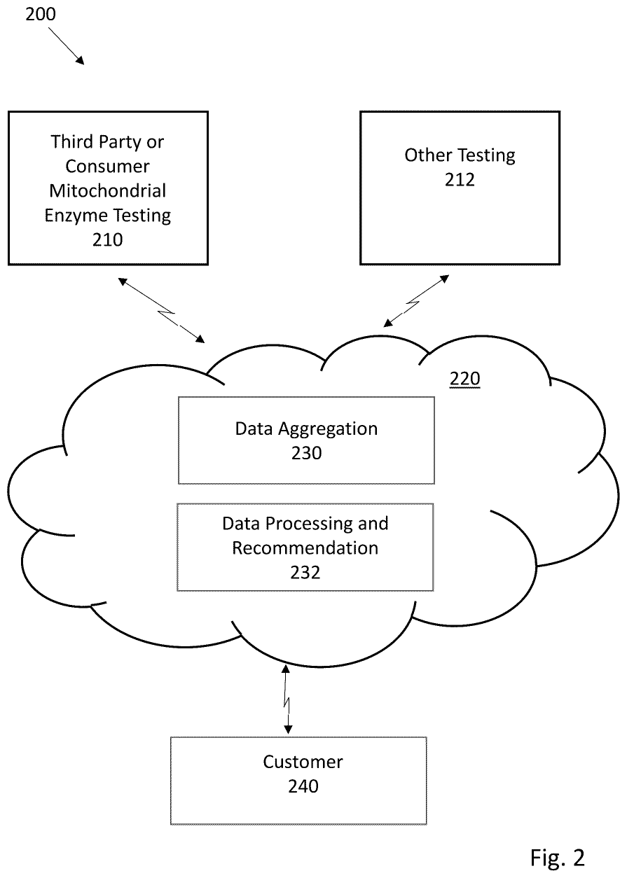

FIG. 2 is an illustration of one embodiment of a recommendation system 200 based on tested mitochondrial enzyme function. In response to customer need, third party or consumer mitochondrial enzyme testing providers 210, with or without optional other testing 212, can supply custom data to a data processing cloud 220. A data aggregation module 230 can collect, filter, and reformat data that is provided to a data processing and recommendation module 232. These recommendations can be supplied to customer 240.

In one embodiment, the third party or consumer mitochondrial enzyme testing provider 210 can be a commercial testing provider. The provider can receive a request for testing via cloud, email, text, chat, social media, customer support site, or other messaging system. The commercial testing provider can send a kit to the customer with necessary sampling equipment. This can include but is not limited to a sampling swab that a customer can use to swab their cheek for placement in a sampling vial. The commercial testing provider can process the sample swab, testing for enzymatic activity of a respectively citrate synthase, Complex I, Complex II, Complex II plus Complex III, and Complex IV. Each of measurements Complex I, Complex II, Complex II plus Complex III, and Complex IV can be normalized to citrate synthase, and all five measurements can be expressed as percent of a mean of a control dataset.

In one embodiment, other testing 212 can include consumer or third-party lab testing. Testing can include body temperature, weight tracking, or tracking of metabolic indicators such as pulse rate, cardiac rhythm, blood oxygen, or blood glucose. Other trackable indicators can include volatile organic compounds (VOC), hydrogen, water vapor or carbon monoxide. In other embodiments, lifestyle trackers that quantify activity, distance walked, or sleep patterns, for example, can be used to refine health related suggestions by the data processing and recommendation module 232. In some embodiments, testing information can be supplemented with information that can include chronological, environmental, or biometric data. For example, work stress can be monitored by tracking frequency or duration of calendared work events. Other indicators can include travel, season, holidays, weekend events, restaurant bills, types of food purchased, or food intake tracking lists.

In some embodiments, the customer 240 can interact with data processing cloud 220 and connected testers 210 and 212 via input to a separate computing device such as a smartphone, table, laptop or computer having a separate processing, memory, and display system. Interaction can be provided by wireless or wired network interface. Input can be through a touchpad, by voice control, or by typing. The display can be a conventional OLED or LCD, or other suitable display. In some embodiments, audio feedback can be provided instead or in addition to visual display. Typically, a user interface is accessible by the user through a smartphone or tablet application such as are provided for Android™ or iPhone™ applications.

Optionally, data and control signals can be received, generated, or transported between varieties of external data sources, including wireless networks or personal area networks, cellular networks, or internet or cloud mediated data sources. In addition, local data storage (e.g. a hard drive, solid state drive, flash memory, or SRAM) can allow for data storage of user-specified preferences or protocols. Data can be immediately presented, stored for later usage and presentation, further combined with aggregated data, or sent onward to a cloud or server for personal update and storage. In one possible embodiment, multiple communication systems can be provided. For example, connections to system 200 and cloud 220 can be provided with a direct Wi-Fi connection (802.11b/g/n), as well as a separate 4G or 5G cell connection provided as a back-up communication channel.

Because data is health related and may be personally sensitive, interaction with data processing cloud 220, and connected testers 210 and 212 can require secure identification of a customer through possession of a designated device, through passwords or biometric authentication, or by other suitable enrollment and authentication procedures. Typically, a customer will have an identifying password that is used in conjunction with a password protected smartphone, tablet, or computer.

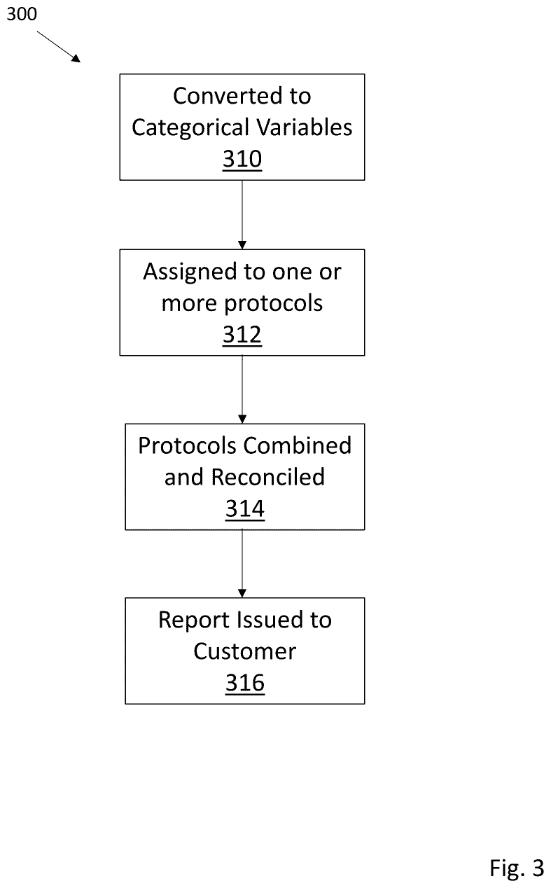

FIG. 3 is flow chart 300 illustrating steps in a recommendation method. In a first step, mitochondrial enzyme testing data is converted to categorical variables 310. In a second step, the data is assigned to one or more protocols 312. In a third step, the protocols are combined and reconciled. In a fourth step 316, a report with recommendations is issued to a customer.

In one embodiment, step 310 further involves taking five received and normalized measurements of citrate synthase, Complex I, Complex II, Complex II plus Complex III, and Complex IV and converting them from a quantitative to a qualitative score. For example, this can result in measurement category variables expressed as Low: ≤50%, Low-Normal: 50-70%, Normal: 70-140%, High-Normal 140-200%, and High: ≥200%.

In one embodiment, the second step 312 can involve noting presence of low or low-normal values for any of the five measurements, as well as other patterns found in their combinations. These patterns can then be used as a series of triggers to include protocols, some but not all of which are mutually exclusive. These protocols can include at least some of, but are not limited to the following twelve protocols:
Fix Complex I protocol—Implemented when Complex I is low or low-normal. In this protocol, riboflavin, iron, and sulfur nutrients are recommended. Drugs such as metformin or berberine are discouraged, as is nylon clothing due to potential leachants such as caprolactam.
Fix Complex II protocol—Implemented when Complex II is low or low-normal. In this protocol, riboflavin, iron, and sulfur nutrients are recommended. Drugs such as antifungal ketoconazole and the anti-tuberculosis medication isoniazid are discouraged, as is exposure to the industrial chemical hydrazine.
Fix Complex II plus III protocol—Implemented when Complex II+III is low or low-normal. In this protocol, supplemental nutrients including CoQ10, vitamin C, vitamin K2 in the form of MK-4, sulfur amino acids, near infrared light in the 700-1000 nanometer range, methylene blue, and the removal of complex III inhibitors from diet, lifestyle practices, and medications are encouraged.
Fix Complex IV protocol—Implemented when Complex IV is low or low-normal. In this protocol, iron, copper, methylene blue, and fumarate are recommended. Drugs such as the antibiotics cefazolin and cefalotin, and the breast cancer drug tamoxifen, are discouraged, as is exposure to hydrogen sulfide, nitric oxide, and carbon monoxide gasses.
Feed Complex II protocol—Implemented when Complex II is ≥1.75× higher than complex II. In this protocol, supplemental nutrients such as succinate, alpha-ketoglutarate, and glutamine are encouraged. Also encouraged is a low-carbohydrate, high-fat, ketogenic diet. Typically, this diet includes restricting intake of starches like bread, pasta, whole grains, legumes (lentils, peas, and beans), fruit, root vegetables like potatoes and carrots, fruits, fruit juices, and added sugars. In place of these foods, coconut, macadamia nuts, avocados, egg yolks, fatty meats, and added fats and oils like MCT oil, butter, and olive oil can be incorporated.
Feed Complex I protocol—Implemented when Complex I is ≥1.75× higher than complex II. In this protocol, the supplemental nutrient glycine is encouraged. Also encouraged is a low-fat, high-carbohydrate diet. Typically, this diet includes starches like soured, sprouted, or fermented whole grains; legumes (peas, sprouted lentils, and sprouted beans), whole fruits, root vegetables like potatoes and carrots, and moderate use of whole food-based sugars like honey and maple syrup. Nuts, coconuts, and added fats and oils like butter, olive oil, coconut oil, or avocado oil should be restricted. In general, leaner cuts of meat, like white fish, poultry, or top sirloin are encouraged.
Peroxisome protocol—Implemented if low or low-normal complex II+III and IV with low complex I that has ≥1.75× the deficit of II+III and IV, but normal or high complex II. In this protocol peroxisome health is promoted with supplemental nutrients such as Docosahexaenoic acid (DHA), Vitamin A, plasmalogens, MCT oil, and L-arginine.

Methylation protocol—Implemented if all complexes except complex II are low with <1.5× magnitude differences between any two of the complexes flagged low and between any low complex and the mean of the low complexes. In this protocol, supplemental nutrients creatine, phosphatidylcholine, trimethylglycine (TMG), thiamin, riboflavin, niacin, B6, folate, B12, magnesium, and zinc are encouraged.
Iron-Sulfur protocol—Implemented if normal or high complex IV is found and all other complexes are low or low-normal. In this protocol, iron, cysteine, thiamin, riboflavin, niacin, B6, magnesium, zinc, vitamin D, glutathione, succinate, and fumarate are recommended. Drugs such as ketoglutarate, vitamin C supplements, antabuse (disulfuram, used for alcoholism), the chemotherapy drug cisplatin, auranofin (used for rheumatoid arthritis) and doxorubicin are discouraged. Also discouraged is exposure to zinc, the herbicide paraquat, the fish poison Fintrol (antimycin A), gallium (from industrial exposure during the production of electronics, some medical applications, or from ski wax, and nitric oxide (from inflammation or from vehicle exhaust or smoke). Conditions that lead to a sedentary lifestyle, or lead to inflammation and oxidative stress should also be avoided. For some, high-intensity interval exercise, simulated altitude training, or high-altitude residence can be helpful.
Heme protocol—Implemented if heme deficiency pattern is found. In this protocol, the supplemental nutrients iron, zinc, copper, riboflavin, vitamins A and D, glycine, tryptophan, B6, sodium, potassium, calcium, sulfate, succinate, and fumarate are recommended. Drugs such as alpha-ketoglutarate, vitamin C supplements, the anti-tuberculosis medicine isoniazid, and the antibiotic chloramphenicol are discouraged. Conditions that lead to alcoholism should also be avoided. For some, high-intensity interval exercise, simulated altitude training, or high-altitude residence can be helpful.
Cytoxan protocol—Implemented if low citrate synthase and low or low normal complexes I and II are found and a customer reports using Cytoxan in a survey. In this protocol, time is needed to heal after Cytoxan use that damages mitochondria. Healing time can be reduced by a fasting-feeding reset, where cardio training and compounds like rapamycin or urolithin A are used to accentuate the fasting state. After fasting, a refeeding state accentuated with a high-protein, high-carbohydrate, high-calorie diet and supplements of S-adenosyl-methionine (SAMe), L-arginine, and L-leucine is useful.
Feed Complex IV protocol—Implemented if complex IV is ≥1.75× higher than control mean and then complex I and then complex II+III, but it is also true that the “Fix Complex II+III protocol” has not been activated. In this protocol, the supplemental nutrients vitamin C, vitamin K2 in the MK-4 form, sulfur amino acids, riboflavin, CoQ10, glutathione, and molybdenum are recommended. Also recommended is near-infrared light in the 700-1000 nanometer range.

In some embodiments, a Mitochondrial Biogenesis protocol is recommended by the data processing and recommendation module. Commonly, mitochondrial biogenesis is included when citrate synthase is low or low normal, which then allows for any other protocols to be included if the results are low without normalization to citrate synthase, and the mitochondrial biogenesis protocol itself includes exercise and supplementation with ubiquinol, resveratrol, nicotinamide riboside, trimethylglycine, urolithin A, and pyrroloquinoline quinone.

In some embodiments, a Sulfur Metabolism Optimization protocol is recommended by the data processing and recommendation module. Commonly, the Sulfur Metabolism Optimization is recommended when complex IV is elevated and includes recommendations to collect additional data with data-dependent decisions that impact the final protocol, with the overall components of the protocol possibly including restriction of methyl donors and sulfur-rich foods and supplements, inclusion of raw apples, titration of carbohydrate, and use of molybdenum, ubiquinol, riboflavin, taurine, and glutathione.

In some embodiments, an Excessive Hypoxia protocol is recommended by the data processing and recommendation module. Commonly, Excessive Hypoxia is recommended when complex II is low and includes adjusting altitude and oxygen availability and possible restriction of succinate and fumarate and/or use of alpha-ketoglutarate, providing other contraindications are not present.

In one embodiment, the third step 314 can involve removal of duplicate protocol elements. If needed, protocols recommendations can be reordered and suggestions rearranged. For example, when “Fix Complex II” and “Fix Complex I” are both included together, a “medications/inhibitors” sections for both of them can be included first, with other recommendations or suggested lifestyle strategies listed thereafter.

In one embodiment, the fourth step 316 can involve other additions to the customer report. In some embodiments, report elements can include a presentation of a customer's raw results, an introduction to general mitochondrial health, suggestions to implement the protocol(s), and a list of other things that may help.

Many modifications and other embodiments of the invention will come to the mind of one skilled in art having the benefit of the teachings presented in the foregoing descriptions and the associated drawings. Therefore, it is understood that the invention is not to be limited to the specific embodiments disclosed, and that modifications and embodiments are intended to be included within the scope of the appended claims. It is also understood that other embodiments of this invention may be practiced in the absence of an element/step not specifically disclosed herein.

What is claimed is:

1. A nutrition recommendation system incorporating results from mitochondrial enzyme testing, comprising:
a data aggregation module arranged to receive mitochondrial enzyme test data from at least one of citrate synthase, Complex I, Complex II, Complex II plus Complex III, and Complex IV testing; and
a data processing and recommendation module that receives the test data and recommends at least one protocol comprising nutrient suggestions for mitochondrial health.

2. The nutrition recommendation system incorporating results from mitochondrial enzyme testing of claim 1, wherein each of measurements Complex I, Complex II, Complex II plus Complex III, and Complex IV can be normalized to citrate synthase.

3. The nutrition recommendation system incorporating results from mitochondrial enzyme testing of claim 1, wherein each of measurements of citrate synthase, Complex I, Complex II, Complex II plus Complex III, and Complex IV can be expressed as percent of a mean of a control dataset.

4. The nutrition recommendation system incorporating results from mitochondrial enzyme testing of claim 1, wherein a Fix Complex I protocol is recommended by the data processing and recommendation module.

5. The nutrition recommendation system incorporating results from mitochondrial enzyme testing of claim 1, wherein a Fix Complex II protocol is recommended by the data processing and recommendation module.

6. The nutrition recommendation system incorporating results from mitochondrial enzyme testing of claim 1, wherein a Fix Complex II plus Complex III protocol is recommended by the data processing and recommendation module.

7. The nutrition recommendation system incorporating results from mitochondrial enzyme testing of claim 1, wherein a Fix Complex IV protocol is recommended by the data processing and recommendation module.

8. The nutrition recommendation system incorporating results from mitochondrial enzyme testing of claim 1, wherein a Feed Complex II protocol is recommended by the data processing and recommendation module.

9. The nutrition recommendation system incorporating results from mitochondrial enzyme testing of claim 1, wherein a Feed Complex I protocol is recommended by the data processing and recommendation module.

10. The nutrition recommendation system incorporating results from mitochondrial enzyme testing of claim 1, wherein a Peroxisome protocol is recommended by the data processing and recommendation module.

11. The nutrition recommendation system incorporating results from mitochondrial enzyme testing of claim 1, wherein a Methylation protocol is recommended by the data processing and recommendation module.

12. The nutrition recommendation system incorporating results from mitochondrial enzyme testing of claim 1, wherein an Iron-Sulfur protocol is recommended by the data processing and recommendation module.

13. The nutrition recommendation system incorporating results from mitochondrial enzyme testing of claim 1, wherein a Heme protocol is recommended by the data processing and recommendation module.

14. The nutrition recommendation system incorporating results from mitochondrial enzyme testing of claim 1, wherein a Cytoxan protocol is recommended by the data processing and recommendation module.

15. The nutrition recommendation system incorporating results from mitochondrial enzyme testing of claim 1, wherein a Feed Complex IV protocol is recommended by the data processing and recommendation module.

16. The nutrition recommendation system incorporating results from mitochondrial enzyme testing of claim 1, wherein a Mitochondrial Biogenesis protocol is recommended by the data processing and recommendation module.

17. The nutrition recommendation system incorporating results from mitochondrial enzyme testing of claim 1, wherein a Sulfur Metabolism Optimization protocol is recommended by the data processing and recommendation module.

18. The nutrition recommendation system incorporating results from mitochondrial enzyme testing of claim 1, wherein an Excessive Hypoxia protocol is recommended by the data processing and recommendation module.

19. A nutrition recommendation method incorporating results from mitochondrial enzyme testing, comprising the steps of:
arranging a data aggregation module to receive mitochondrial enzyme testing test data from at least one of citrate synthase, Complex I, Complex II, Complex II plus Complex III, and Complex IV testing; and
using a data processing and recommendation module to recommend at least one protocol comprising nutrient suggestions for mitochondrial health.

20. The nutrition recommendation method incorporating results from mitochondrial enzyme testing of claim 19, wherein each of measurements Complex I, Complex II, Complex II plus Complex III, and Complex IV can be normalized to citrate synthase.

21. The nutrition recommendation method incorporating results from mitochondrial enzyme testing of claim 19, wherein each of measurements of citrate synthase, Complex I, Complex II, Complex II plus Complex III, and Complex IV can be expressed as percent of a mean of a control dataset.
